(19) United States
(12) Patent Application Publication
(10) Pub. No.: US 2006/0286625 A1
(43) Pub. Date: Dec. 21, 2006

(54) Rapid enrichment for Listeria

(75) Inventors: Meghan A. McIlroy, Des Plaines, IL (US); David K. Hayashi, Chicago, IL (US)

Correspondence Address: FITCH EVEN TABIN & FLANNERY, 120 S. LASALLE STREET, SUITE 1600, CHICAGO, IL, 60603-3406, US

(73) Assignee: Kraft Foods Holdings, Inc.

(21) Appl. No.: 11/152,923

(22) Filed: Jun. 15, 2005

(51) Int. Cl. C12Q 1/04; C12N 1/20

(52) U.S. Cl. 435/34; 435/253.6

(57) ABSTRACT

A non-selective and selective media and method are provided for the rapid enrichment of Listeria from samples. The method includes inoculating a sample that may contain Listeria into a non-selective or selective medium. Also, a sample that may contain Listeria may be provided by an environmental sponge or swab. More specifically, a small volume of the medium is added directly to an environmental sample for one-step enrichment in a short time period. This invention enables shorter enrichment time, as genetic detection methods become more rapid and sensitive.

The invention provides selective and non-selective media and methods for the rapid enrichment of Listeria from samples. More specifically, a small volume of the medium is added directly to an environmental sample for one-step enrichment in a short time period. The selective medium includes antimicrobial agents that favor the growth of Listeria spp. while inhibiting the growth of other microflora.

### BACKGROUND

Listeria is classified as gram-positive, rod-shaped bacteria and consists of the species Listeria monocytogenes, L. innocua, L. welshimeri, L. seeligeri, L. ivanovii, and L. grayi. Among these, L. monocytogenes is responsible for the majority of human listeriosis cases and immunocompromised, pregnant women, elderly, and newborns have increased susceptibility to infection. The most common symptoms of listeriosis are septicemia, meningitis, and miscarriages.

A large number of methods for detecting Listeria are known. Conventional detection methods for L. monocytogenes comprise of pre-enriching and subsequently isolating colonies on selection media (Lovett et al., J. Food Protection 50 (1987), 188-192; McClain & Lee, J. Assoc. Off. Anal. Chem. 71 (1988), 660-664). Single colonies are examined for their morphology and biochemical properties. An analysis may take up to 6-8 days.

Some current industry accepted Listeria enrichments require a 24 to 48 hour incubation time with one to two enrichment steps. Many of the media formulations are selective and can be inhibitory towards Listeria spp. Often, selective agents do not support the resuscitation of injured organisms. As genetic detection methods become more rapid and sensitive, shorter and simpler enrichment is needed. In order to achieve an improved enrichment method, an enrichment medium should sustain an increased growth rate, resuscitate injured cells, and either inhibit growth of competitor organisms or allow Listeria spp. to grow to detectable levels through a competitive environment in a short time period.

Rapid, one-step enrichment for Listeria is needed. This invention provides such methods.

### SUMMARY

Selective and non-selective Listeria spp. enrichment media are provided. Both media contain sources of protein, carbohydrates, vitamins, minerals, buffer and essential ions. In an important aspect, both media contain pyruvate in amounts effective for stimulating the metabolism of stressed organisms. The selective medium further includes antimicrobial agents that favor the growth of Listeria spp. while inhibiting the growth of other microflora. The enrichment media are effective for maximizing the growth of Listeria spp. over a ten to twelve hour incubation time. In this aspect, both selective and non-selective media are effective for supporting minimally 2 logs of Listeria spp. growth after 10 hours at 30° C.

The non-selective enrichment medium for Listeria includes the following components:

| Component | Range g/L | Preferred g/L |
|---|---|---|
| Pyruvate | 2.0-10.0 | 2.5 |
| Carbohydrate | 0.2-2.5 | 0.5 |
| Yeast Extract | 1.0-10.0 | 3.0 |
| Sodium Chloride | 1.0-5.0 | 5.0 |
| Magnesium Source | 0.1-0.5 | 0.25 |
| Iron Source | 0.1-2.5 | 0.1 |
| Protein | 1.0-20.0 | 10.0 |
| Buffer | 1.0-25.0 | Sufficient amount to |
| | | maintain pH 6.0-7.3 during |
| | | incubation. |

The selective enrichment medium for Listeria includes the following components:

| Component | Range g/L | Preferred g/L |
|---|---|---|
| Pyruvate | 2.0-10.0 | 2.5 |
| Carbohydrate | 0.2-2.5 | 0.5 |
| Yeast Extract | 1.0-10.0 | 3.0 |
| Sodium Chloride | 1.0-5.0 | 5.0 |
| Magnesium Source | 0.1-0.5 | 0.25 |
| Iron Source | 0.1-2.5 | 0.1 |
| Polymyxin B | 0.007-0.06 | 0.007-0.025 |
| Acriflavine HCl | 0.004-0.03 | 0.004-0.015 |
| Ceftazidime | 0.015-0.12 | 0.015-0.045 |
| Protein | 1.0-20.0 | 10.0 |
| Buffer | 1.0-25.0 | Sufficient amount to |
| | | maintain pH 6.0-7.3 |
| | | during incubation |

Pyruvate is included in the media to enhance growth and recovery of stressed cells. The pyruvate is provided to the medium as a salt of pyruvic acid, preferably a sodium salt of pyruvic acid. In an important aspect, the medium includes from about 2.0 to about 10.0 g/L sodium pyruvate.

The medium further includes carbohydrate such as dextrose, esculin, maltose, amygdalin, cellobiose, fructose, mannose, salicin, dextrin, (x-methyl-D-glucoside and mixtures thereof. In an important aspect, the medium includes from about 0.2 to about 2.5 g/L dextrose.

The medium further includes Yeast Extract. In an important aspect, the medium includes from about 1.0 to about 10.0 g/L Yeast Extract.

The medium further includes salts such as sodium, potassium, or calcium salts of chloride. In an important aspect, the medium includes from about 1.0 to about 5.0 g/L sodium chloride.

Essential ions such as magnesium and iron are also included in the media. Magnesium which may be used including magnesium selected from the group consisting of magnesium sulfate, magnesium chloride, and mixtures thereof. Iron which may be used inludes iron selected from the group consisting of ferric ammonium citrate, ferrous sulfate, ferric sulfate, ferric citrate, ferrous ammonium sulfate, ferric chloride, and mixtures thereof. In an important aspect, the medium includes from about 0.1 to about 0.5 g/L magnesium sulfate heptahydrate and from about 0.1 to about 2.5 g/L ferric ammonium citrate.

The medium further includes protein, which may be provided from a variety of sources. For example, the protein may be provided from sources such as Tryptone, Tryptose, Soytone, Peptone, Pantone, Bitone, Proteose Peptone, pancreatic digest of gelatin, and mixtures thereof. In an important aspect, the medium includes from about 1.0 to about 20.0 g/L protein.

The medium further includes buffers, which are effective for maintaining the pH in a desired range. For example, buffers that may be used include buffers such as potassium phosphate monobasic, potassium phosphate dibasic, sodium phosphate dibasic, and mixtures thereof. In an important aspect, the medium includes from about 1.0 to about 25.0 g/L buffer.

The selective medium further comprises of antimicrobial agents. Antimicrobials that may be used in the present invention include selective agents from the group consisting of Polymyxin B, Acriflavine HCl, Ceftazidime, and mixtures thereof. In an important aspect, the medium includes from about 0.007 to about 0.06 g/L Polymyxin B, from about 0.004 to about 0.03 g/L Acriflavine HCl, and from about 0.015 to about 0.12g/L Ceftazidime.

An enrichment method for Listeria is also provided that includes inoculating a sample which may contain Listeria into a non-selective or selective medium as described. Enrichment in non-selective or selective medium may be practiced with a food sample, which may contain Listeria. Also, a sample which may contain Listeria may be provided by an environmental sponge or swab. The environmental sponge or swab may include a medium selected from the group consisting of buffered peptone water.

### BRIEF DESCRIPTION OF THE FIGURE

FIG. 1 illustrates Listeria growth at 30° C. as compared to commercially available media.

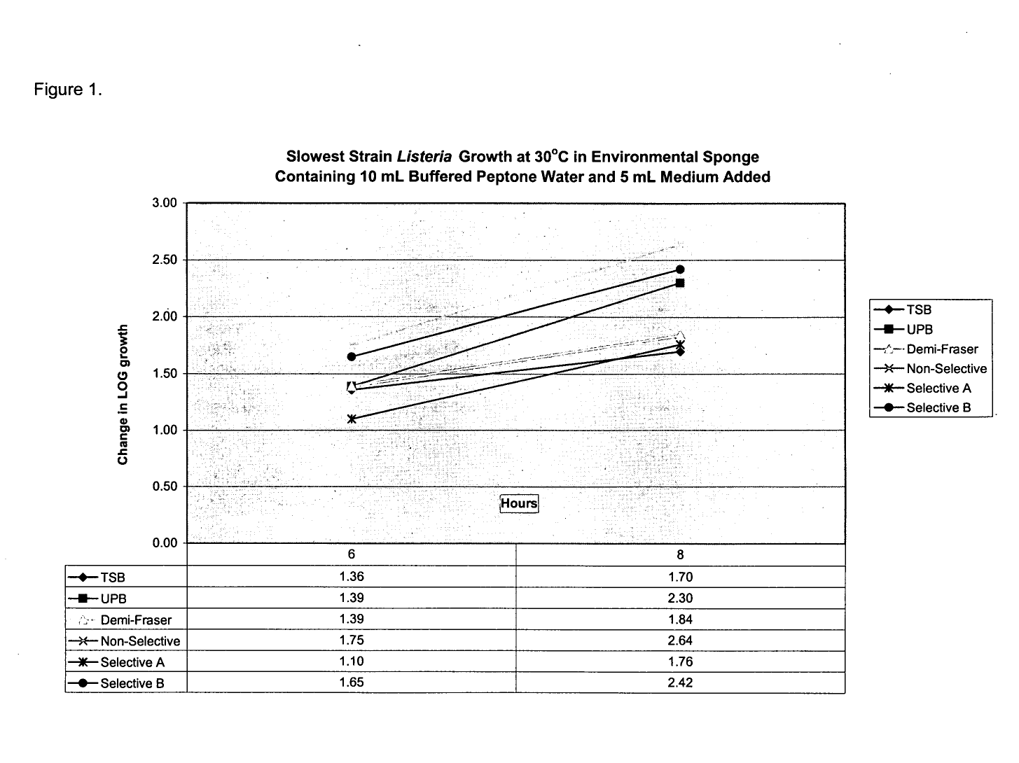

### DETAILED DESCRIPTION

Definitions

The term “Listeria” as used herein, refers to the bacteria classified as such in Bergey's Manual of Systematic Bacteriology (P. H. A. Sneath (ed), 1986, 1234-1245, Williams & Wilkins). Therefore, the term “Listeria” as used herein includes Listeria monocytogenes, Listeria innocua, Listeria seeligeri, Listeria welshimeri, Listeria ivanovii, and Listeria grayi.

“Non-selective” medium does not contain additives that intentionally inhibit the growth of any organisms picked up by a swab or sponge. Other organisms in addition to Listeria are capable of growing in this medium. The media components are optimized to achieve maximum resuscitation and growth of Listeria spp.

“Selective” medium contains additives that inhibit many organisms' growth other than Listeria spp. The “selective” medium is designed to maximize the growth of Listeria spp. and reduce background microflora. There are several microorganisms that are unaffected by the selective components so not all non-Listeria organisms are inhibited. The medium is not selective for different Listeria species since the objective is to enrich for all Listeria.

Medium Preparation

Both the selective and non-selective media may be prepared using known methods. Generally, all ingredients are weighed out in deionized water and filter sterilized using a 0.2 μm (Nalgene) filter or autoclaved at 121° C. for 15 minutes. Selective agents are reconstituted with sterile water and added just prior to use of the medium.

Sample Preparation

Samples to be tested for the presence of Listeria spp. may be added directly to selective and non-selective media or may be collected on swabs or sponges. Pre-hydrated sponges and swabs are used to collect microorganism according to manufacturer's instructions and placed back in original container. Selective or non-selective medium is added directly to the container, vortexed or stomached for 10 seconds, and incubated at 30-37° C. Small volumes of medium, such as about 30 ml or less, preferably about 5 ml, may be added to the swab or sponge in preparation for the enrichment.

### EXAMPLES

### Example 1

### Media Formulations

The g/L (total) column is the composition of the media and the Buffered Peptone Water (BPW) combined if 5 ml of medium is added to a sponge or swab containing 10 ml BPW. The right column is the composition for the medium considered independently from BPW. In this example, the buffer and protein source are supplied by the BPW, but if growth and buffering capacity are insufficient, the medium may be supplemented with additional protein and buffer.

| | g/L (total) | g/L |
|---|---|---|
| NON-SELECTIVE MEDIUM | | |
| Sodium Chloride | 1.667 | 5.00 |
| Dextrose | 0.500 | 1.50 |
| Magnesium Sulfate 7H₂0 | 0.250 | 0.75 |
| Ferric Ammonium Citrate | 0.100 | 0.30 |
| Sodium Pyruvate | 2.500 | 7.50 |
| Yeast Extract | 3.000 | 9.00 |
| Deionized Water | 333.333 | 1000.00 |
| INGREDIENTS FROM BPW IN | | |
| SPONGE/SWAB | | |
| Pancreatic Digest of Gelatin | 6.667 | 10.0 |
| Sodium Chloride | 3.333 | 5.0 |
| Disodium Phosphate | 2.333 | 3.5 |
| Monopotassium Phosphate | 1.000 | 1.5 |
| Deionized Water | 666.667 | 1000 |
| SELECTIVE MEDIUM “A” | | |
| Sodium Chloride | 1.667 | 5.14 |
| Dextrose | 0.500 | 1.54 |
| Magnesium Sulfate 7H₂0 | 0.250 | 0.77 |
| Ferric Ammonium Citrate | 0.100 | 0.31 |
| Sodium Pyruvate | 2.500 | 7.71 |
| Yeast Extract | 3.000 | 9.25 |
| Deionized Water | 324.333 | 1000.00 |
| Polymyxin B | 0.023 | 2.50 |
| Acriflavine HCl | 0.011 | 1.25 |
| Ceftazidime | 0.045 | 5.00 |
| Sterile Deionized Water | 9.000 | 1000.00 |
| INGREDIENTS FROM BPW IN | | |
| SPONGE/SWAB | | |
| Pancreatic Digest of Gelatin | 6.667 | 10.0 |
| Sodium Chloride | 3.333 | 5.0 |
| Disodium Phosphate | 2.333 | 3.5 |
| Monopotassium Phosphate | 1.000 | 1.5 |
| Deionized Water | 666.667 | 1000 |
| SELECTIVE MEDIUM “B” | | |
| Sodium Chloride | 1.667 | 5.05 |
| Dextrose | 0.500 | 1.51 |
| Magnesium Sulfate 7H₂0 | 0.250 | 0.76 |
| Ferric Ammonium Citrate | 0.100 | 0.30 |
| Sodium Pyruvate | 2.500 | 7.57 |
| Yeast Extract | 3.000 | 9.08 |
| Deionized Water | 330.333 | 1000.00 |
| Polymyxin B | 0.008 | 2.50 |
| Acriflavine HCl | 0.004 | 1.25 |
| Ceftazidime | 0.015 | 5.00 |
| Sterile Deionized Water | 3.000 | 1000.00 |
| INGREDIENTS FROM BPW IN | | |
| SPONGE/SWAB | | |
| Pancreatic Digest of Gelatin | 6.667 | 10.0 |
| Sodium Chloride | 3.333 | 5.0 |
| Disodium Phosphate | 2.333 | 3.5 |
| Monopotassium Phosphate | 1.000 | 1.5 |
| Deionized Water | 666.667 | 1000 |

### Example 2

### Comparison of Growth of Listeria monocytogenes in Non-Selective and Selective Media Compared to Commercially Available Media in Environmental Sponges

The slowest growing strain in a collection of Listeria was used. Listeria monocytogenes (DUP-1038, Serotype 4b) was inoculated into Brain Heart Infusion broth (BHI, Difco) and incubated for 24 h at 30° C. to reach stationary phase. The cultures were held at 4° C. for 5 days after reaching stationary phase. To determine the growth level after refrigeration, the culture was serially diluted in Butterfield's Buffer (International BioProducts), pour-plated with Oxford Medium Base (Difco), and incubated at 35° C. for 24 h. Based on the cell counts, the culture was diluted in Butterfield's Buffer and a volume of the diluted culture targeting an initial cell count of 10 CFU/sponge was inoculated into environmental sponges containing Buffered Peptone Water (International BioProducts). In addition, the inoculum was pour plated with Oxford Medium Base. The plates were incubated at 35° C. for 24 h and the cell counts were collected to confirm the CFU/sponge at T₀.

Spiked sponges were processed by adding 5 ml of a medium to the sponge in the original manufacturer's container, followed by stomaching for 10 seconds, and incubation for 10 hours at 30° C. The sponges were stomached again for 10 seconds and serial dilutions were pour-plated with Oxford Medium Base. The plates were incubated at 35° C. for 24 h and the cell counts were collected and compared to the To count at 8 h and 10 h.

Commercial media were compared to Non-Selective and Selective media listed in Example 1. Commercial media tests consisted of Tryptic Soy broth (TSB, Difco), Universal Preenrichment broth (UPB, Difco), and Demi-Fraser Broth Base (DF, Difco)

TABLE 1
Change in Log CFU/ml Listeria monocytogenes during
10-hour incubation at 30° C. in environmental sponges

| Media | 8 h | 10 h |
|---|---|---|
| TSB | 1.36 | 1.70 |
| UPB | 1.39 | 2.30 |
| Demi-Fraser | 1.39 | 1.84 |
| Non-Selective | 1.75 | 2.64 |
| Selective “A” | 1.10 | 1.76 |
| Selective “B” | 1.65 | 2.42 |

Results show Non-Selective medium supports higher growth of Listeria monocytogenes than the commercially available media tested. Selective “A” medium contains the highest concentration of selective agents in the preferred range and performs similarly to Demi-Fraser, a commercially acceptable selective medium often used for the first step in a two-step Listeria enrichment. Selective “B” medium contains the lowest concentration of the selective agents in the preferred range and performs slightly better than UPB.

### Example 3

### Comparison of Growth of Listeria and Environmental Isolates to Determine the Effect of Environmental Isolates on Listeria Growth and the Effect of the Polymyxin B, Acriflavine HCl, and Ceftazidime on Environmental Isolates

Nineteen non-Listeria organisms isolated from environmental sponges from meat and dairy manufacturing facilities were inoculated separately into Tryptic Soy broth (TSB, Difco) and incubated for 24 h at 30° C. The isolates were screened for growth in Selective and Non-Selective media by monitoring absorbance via a spectrophotometer. 900 μl of test media and either 100 μl of culture or Butterfield's Buffer (International BioProducts) were added to a cuvette and incubated at 30° C. Absorbance readings were taken at 660 nm on a spectrophotometer (Shimadzu UV160U) at To, 2 h, 4 h, and 6 h and compared to the corresponding blank cuvette with buffer. Growth was scored by absorbance readings.

The Non-Selective medium consisted of Universal Preenrichment Broth (UPB, Difco) with 0.4% Yeast Extract (YE, Difco). The Selective medium consisted of Universal Preenrichment Broth (UPB, Difco) with 0.4% Yeast Extract (YE, Difco), 0.001% Polymymin B, 0.0005% Acriflavine HCl, and 0.002% Ceftazidime.

TABLE 1
Scoring of Environmental Isolates Based on Absorbance Readings
over 6 hours of Incubation at 30° C.

| | 30° C. | | 35° C. | |
|---|---|---|---|---|
| | | Non- | | Non- |
| Environmental Organism | Selective | Selective | Selective | Selective |
| Serratia | ++ | +++ | ++ | +++ |
| Citrobacter | 0 | +++ | 0 | +++ |
| Enterobacter | ++ | +++ | 0 | +++ |
| Pseudomonas | ++ | +++ | 0 | ++ |
| Pantoea | + | ++ | 0 | ++ |
| Vibrio | − | + | 0 | 0 |
| Acinetobacter | − | + | 0 | + |
| Escherichia coli | 0 | +++ | − | +++ |
| Carnobacterium | ++ | ++ | ++ | ++ |
| Bacillus subtilis | 0 | + | 0 | + |
| Bacillus lichenformis | + | ++ | 0 | ++ |
| Lactococcus | ++ | +++ | ++ | +++ |
| Lactobacillus plantarum | − | + | + | + |
| Lactobacillus casei | − | + | + | + |
| Enterococcus | ++ | ++ | ++ | ++ |
| Staphylococcus | − | +++ | − | +++ |
| epedermis | | | | |
| Staphylococcus aureus | + | +++ | + | +++ |
| Leuconostoc | − | + | 0 | + |
| paramesenteroides | | | | |
| Streptococcus | − | 0 | 0 | 0 |
| thermophilus | | | | |

KEY
− negative absorbance
0 no change in absorbance
+ low growth
++ medium growth
+++ high growth

Several environmental organisms screened via spectrophotometer were selected to test in mixed culture with Listeria. Environmental isolates were inoculated separately into TSB and incubated for 24 h at 30° C. The growth level was determined by serial dilutions plated on Tryptic Soy agar (TSA, Difco) and incubated at 30° C. for 24 h. Based on the cell counts, the culture was diluted in Butterfield's Buffer and a volume of the diluted culture targeting an initial cell count of 1000 CFU/mL was inoculated into the test media.

Listeria monocytogenes (DUP-1038, Serotype 4b) and Listeria welshermeri (DUP-1073) were inoculated separately into Brain Heart Infusion broth (BHI, Difco) and incubated for 24 h at 30° C. to reach stationary phase. The cultures were held at 4° C. for 5 days after reaching stationary phase. To determine the growth level after refrigeration, the culture was serially diluted in Butterfield's Buffer, pour-plated with Oxford Medium Base (Difco), and incubated at 35° C. for 24 h. Based on the cell counts, the culture was diluted in Butterfield's Buffer and a volume of the diluted culture targeting an initial cell count of 100 CFU/mL was inoculated into the test media.

30 mL of Non-Selective or Selective medium was added to 10 ml of Butterfield's Buffer to simulate the addition of media to environment sponge. A Non-Selective and Selective medium was inoculated with Listeria culture alone, environmental isolate alone, and Listeria and environment isolate combined. The media were incubated in a water bath at 30° C. Serial dilutions of inoculated media were collected at To and 8 h and plated on Oxford Media Base and a differential media particular to the environmental isolate being tested (see Table 2). The Oxford Media Base plates were incubated at 35° C. for 24 h and the cell counts were collected and compared to the T₀ count at 8 h.

TABLE 2
Differential Plating Medium

| | | Incubation Temperature |
|---|---|---|
| Isolate | Plating Medium | (° C.) |
| Staphylococcus | Tryptic Soy Agar + 5.0% | 30 |
| aureus | Sheep Blood | |
| Enterobacter | Tryptic Soy Agar + 5.0% | 30 |
| | Sheep Blood | |
| Bacillus subtilis | Tryptic Soy Agar + 5.0% | 30 |
| | Sheep Blood | |
| Enterococcus | Tryptic Soy Agar + 5.0% | 45 |
| | Sheep Blood | |
| Carnobacterium | Tryptic Soy Agar + 1.0% | 30 |
| | Crystal Violet | |

TABLE 3
Growth of Listeria and environmental isolates in Selective and Non-Selective Media

| | Change in | | | |
|---|---|---|---|---|
| | Log CFU/ml Listeria | | | |
| | in 8 hours at 30° C. | | Change in Log CFU/mL | |
| | Non- | | Environmental Isolate | |
| Environmental | Selective | Selective | Non-Selective | Selective |

| | | | | | | |
|---|---|---|---|---|---|---|
| Listeria strain | Isolate | Pure | Mixed | Pure | Mixed | Mixed |

| | | | | | | | |
|---|---|---|---|---|---|---|---|
| L. welshermeri | Staphylococcus | 2.28 | 1.78 | 1.85 | 1.81 | 3.05 | −1.07 |
| | aureus Strain 1 | | | | | | |
| L. welshermeri | Enterobacter | 2.46 | 2.31 | 2.00 | 2.53 | 4.64 | −2.70 |
| L. welshermeri | Bacillus subtilis | 2.10 | 2.19 | 1.55 | 1.88 | 3.11 | −0.78 |
| L. welshermeri | Enterococcus | 2.56 | 2.44 | 2.28 | 2.19 | 3.48 | 2.68 |
| L. welshermeri | Carnobacterium | 2.46 | 2.63 | 1.89 | 1.92 | 2.51 | 1.33 |
| L. monocytogenes | Enterococcus | 1.71 | 1.88 | 1.38 | 1.47 | 3.37 | 1.97 |
| | durans cocktail | | | | | | |
| L. monocytogenes | Staphylococcus | 1.92 | 1.94 | 1.51 | 1.49 | 3.45 | −0.50 |
| | aureus Strain 2 | | | | | | |

Polymyxin B, Acriflavine HCl, and Ceftazidime are effective selective agents against many environmental isolates that have the potential to be included in an environmental sample. At 8 h, Staphylococcus aureus, Enterobacter, Bacillus subtilis CFU/mL were reduced from the CFU/mL at T₀. Enterococcus and Carnobacterium grew in Selective medium, but 0.8-1.2 log less growth than in Non-Selective medium. In most cases, the presence of environmental isolates had minimal effect on the growth of Listeria. In the Non-Selective medium, L. welshermeri showed 0.5 log less growth in the presence of S. aureus Strain 1 than in a pure culture. In the Selective medium, L. welshermeri showed 0.53 and 0.33 log more growth in the presence of Enterobacter and B. subtilis respectively than in a pure culture.

What is claimed is:

1. A non-selective enrichment medium for Listeria spp. comprising an amount of pyruvate effective for supporting at least 2 logs of Listeria spp. growth after 10 hours at 30° C.

2. The non-selective enrichment medium of claim 1 wherein the medium includes from about 2.0 to about 10.0 g/L of a sodium salt of pyruvic acid.

3. The non-selective enrichment medium of claim 1 wherein the medium further includes from about 0.2 to about 2.5 g/L carbohydrate.

4. The non-selective enrichment medium of claim 3 wherein the carbohydrate is dextrose.

5. A non-selective enrichment medium for Listeria comprising:
from about 2.0 to about 10.0 g/L pyruvate;
from about 0.2 to about 2.5 g/L carbohydrate;
from about 1.0 to about 10.0 g/L Yeast Extract;
from about 1.0 to about 5.0 g/L salt;
from about 0.1 to about 0.5 g/L of a magnesium source;
from about 0.1 to about 2.5 g/L of an iron source; and
from about 1.0 to about 20.0 g/L protein,
the medium including an amount of buffer sufficient to maintain pH 6.0-7.3 during incubation.

6. The medium of claim 5 wherein the pyruvate is a sodium salt of pyruvic acid.

7. The medium of claim 5 wherein the carbohydrate provided to the medium as dextrose.

8. The medium of claim 5 wherein the magnesium is selected from the group consisting of magnesium sulfate, magnesium citrate and mixtures thereof.

9. The medium of claim 5 wherein the iron is selected from the group consisting of ferric ammonium citrate, ferrous sulfate, ferric sulfate, ferric citrate, ferrous ammonium sulfate, ferric chloride, and mixtures thereof.

10. An enrichment method for Listeria comprising inoculating a sample which may contain Listeria spp. into a non-selective enrichment medium comprising an amount of pyruvate effective for supporting at least 2 logs of Listeria spp. growth after 10 hours at 30° C.

11. The enrichment method of claim 10 wherein the sample that may contain Listeria is provided by an environmental sponge or swab.

12. The method of claim 11 wherein the environmental sponge or swab includes buffered peptone water.

13. The method of claim 11 wherein about 30 ml of non-selective medium is added to the sponge or swab.

14. A selective enrichment medium for Listeria spp. comprising an amount of pyruvate effective for supporting at least 2 logs of Listeria spp. growth after 10 hours at 30° C. and an antimicrobial agent.

15. The selective enrichment medium of claim 14 wherein the medium includes from about 2.0 to about 10.0 g/L of a sodium salt of pyruvic acid.

16. The selective enrichment medium of claim 14 wherein the medium includes an antimicrobial agent selected from the group consisting of Polymyxin B, Acriflavine HCl, Ceftazidime, and mixtures thereof.

17. The selective medium of claim 16 wherein the medium includes from about 0.007 to about 0.06 g/L Polymyxin B, from about 0.004 to about 0.03 g/L Acriflavine HCl, and from about 0.015 to about 0.12g/L Ceftazidime.

18. The selective enrichment medium of claim 14 wherein the medium further includes from about 0.2 to about 2.5 g/L carbohydrate.

19. The selective enrichment medium of claim 18 wherein the carbohydrate is dextrose.

20. A selective enrichment medium for Listeria comprising:
from about 2.0 to about 10.0 g/L pyruvate;
from about 0.2 to about 2.5 g/L carbohydrate;
from about 1.0 to about 10.0 g/L Yeast Extract;
from about 1.0 to about 5.0 g/L salt;
from about 0.1 to about 0.5 g/L of a magnesium source;
from about 0.1 to about 2.5 g/L of an iron source;
from about 1.0 to about 20.0 g/L protein;
from about 0.007 to about 0.06 g/L Polymyxin B;
from about 0.004 to about 0.03 g/L Acriflavine HCl; and
from about 0.015 to about 0.12 g/L Ceftazidime,
the medium including an amount of buffer sufficient to maintain pH 6.0-7.3 during incubation.

21. The medium of claim 20 wherein the pyruvate is a sodium salt of pyruvic acid.

22. The medium of claim 20 wherein the carbohydrate provided to the medium as dextrose.

23. The medium of claim 20 wherein the iron is selected from the group consisting of ferric ammonium citrate, ferrous sulfate, ferric sulfate, ferric citrate, ferrous ammonium sulfate, ferric chloride, and mixtures thereof.

24. The medium of claim 20 wherein the magnesium is selected from the group consisting of magnesium sulfate, magnesium citrate, and mixtures thereof.

25. An enrichment method for Listeria comprising inoculating a sample which may contain Listeria spp. into a selective enrichment medium comprising an amount of pyruvate effective for supporting at least 2 logs of Listeria spp. growth after 10 hours at 30° C. and a antimicrobial agent.

26. The enrichment method of claim 25 wherein the sample that may contain Listeria is provided by an environmental sponge or swab.

27. The enrichment method of claim 26 wherein the environmental sponge or swab includes buffered peptone water.

28. The enrichment method of claim 26 wherein about 30 ml or less of selective medium is added to the sponge or swab.
